SEQUENCE LISTING (14 sequences)

SEQ ID NO: 1 - DNA, 18 bp, synthetic construct
  source 1..18: mol_type = other DNA; note = 5' end primer M13-F
tgtaaaacgacggccagt

SEQ ID NO: 2 - DNA, 20 bp, synthetic construct
  source 1..20: mol_type = other DNA; note = 3' end primer KDP004
tcctggggacagtgacagtg

SEQ ID NO: 3 - DNA, 37 bp, synthetic construct
  source 1..37: mol_type = other DNA; note = 5' end primer KDP047
cactgtcactgtccccaggagccacccccctgggccc

SEQ ID NO: 4 - DNA, 22 bp, synthetic construct
  source 1..22: mol_type = other DNA; note = 3' end primer BGH-R
aactagaaggcacagtcgaggc

SEQ ID NO: 5 - DNA, 36 bp, synthetic construct
  source 1..36: mol_type = other DNA; note = 3' end primer KDP048
tggtggtgtctagagactcagggctgggcaaggtgg

SEQ ID NO: 6 - DNA, 1872 bp, synthetic construct
  source 1..1872: mol_type = other DNA; note = Heavy chain G-CSF nucleotide sequence of trastuzumab fusion protein
gaagtgcagctggtcgaatctgggggagggctggtgcagccaggaggatcactgaggctgtcctgcgccgctagcgggttcaacatcaaggacacctacattcactgggtcagacaggctcctggcaagggactggagtgggtggcacgcatctatccaactaatgggtacaccagatatgccgactctgtgaagggtcggtttaccatttctgcagatacaagtaaaaacactgcctacctgcagatgaactccctgcgagccgaagatacagccgtgtactattgcagtcgttgggggggtgacggattctacgctatggattattgggggcagggcaccctggtcacagtgtccagcgcatcaacaaaggggccttccgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcgggaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcagaaatcactgtcactgtccccaggagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 7 - DNA, 642 bp, synthetic construct
  source 1..642: mol_type = other DNA; note = Light chain nucleotide sequence of trastuzumab fusion protein
gacattcagatgactcagtctccttcatcactgtccgctagcgtgggcgacagagtcactatcacctgccgcgcatcccaggatgtgaacaccgcagtcgcctggtatcagcagaagcctggcaaagctccaaagctgctgatctactctgcaagtttcctgtatagtggagtgccctcaaggttttcagggtcccggagcggcaccgacttcacactgactatctccagcctgcagcctgaggattttgccacatactattgccagcagcactataccacaccccctactttcggccagggaaccaaagtggagatcaagcgaactgtggccgctccatctgtcttcatttttccacccagtgacgaacagctgaagtccgggacagctagcgtggtctgtctgctgaacaatttttaccccagggaagccaaagtgcagtggaaggtcgataacgctctgcagtctggaaatagtcaggagtcagtgacagaacaggactccaaagatagcacttattctctgtctagtaccctgacactgagcaaggcagactacgagaagcataaagtgtatgcctgtgaagtcactcatcaggggctgtccagtcccgtcacaaaatcctttaatcgtggcgaatgt

SEQ ID NO: 8 - DNA, 1350 bp, synthetic construct
  source 1..1350: mol_type = other DNA; note = Heavy chain nucleotide sequence of trastuzumab fusion protein
gaagtgcagctggtcgaatctgggggagggctggtgcagccaggaggatcactgaggctgtcctgcgccgctagcgggttcaacatcaaggacacctacattcactgggtcagacaggctcctggcaagggactggagtgggtggcacgcatctatccaactaatgggtacaccagatatgccgactctgtgaagggtcggtttaccatttctgcagatacaagtaaaaacactgcctacctgcagatgaactccctgcgagccgaagatacagccgtgtactattgcagtcgttgggggggtgacggattctacgctatggattattgggggcagggcaccctggtcacagtgtccagcgcatcaacaaaggggccttccgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcgggaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcagaaatcactgtcactgtccccagggaaa

SEQ ID NO: 9 - DNA, 1188 bp, synthetic construct
  source 1..1188: mol_type = other DNA; note = Trastuzumab fusion protein light chain -linker-G-CSF nucleotide sequence
gacattcagatgactcagtctccttcatcactgtccgctagcgtgggcgacagagtcactatcacctgccgcgcatcccaggatgtgaacaccgcagtcgcctggtatcagcagaagcctggcaaagctccaaagctgctgatctactctgcaagtttcctgtatagtggagtgccctcaaggttttcagggtcccggagcggcaccgacttcacactgactatctccagcctgcagcctgaggattttgccacatactattgccagcagcactataccacaccccctactttcggccagggaaccaaagtggagatcaagcgaactgtggccgctccatctgtcttcatttttccacccagtgacgaacagctgaagtccgggacagctagcgtggtctgtctgctgaacaatttttaccccagggaagccaaagtgcagtggaaggtcgataacgctctgcagtctggaaatagtcaggagtcagtgacagaacaggactccaaagatagcacttattctctgtctagtaccctgacactgagcaaggcagactacgagaagcataaagtgtatgcctgtgaagtcactcatcaggggctgtccagtcccgtcacaaaatcctttaatcgtggcgaatgtggcagcggcggtggcggatccgccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 10 - DNA, 21 bp, synthetic construct
  source 1..21: mol_type = other DNA; note = linker
ggcagcggcggtggcggatcc

SEQ ID NO: 11 - protein, 624 aa, synthetic construct
  source 1..624: note = Heavy chain G-CSF amino acid sequence of trastuzumab fusion protein
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 12 - protein, 214 aa, synthetic construct
  source 1..214: note = Light chain amino acid sequence of trastuzumab fusion protein
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 450 aa, synthetic construct
  source 1..450: note = Heavy chain amino acid sequence of trastuzumab fusion protein
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 396 aa, synthetic construct
  source 1..396: note = Amino acid sequence of light chain linker-G-CSF of Trastuzumab fusion protein
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGGGSATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

(19) United States
(12) Patent Application Publication
(10) Pub. No.: US 2025/0136654 A1
(43) Pub. Date: May 1, 2025

(54) ANTI-HER-2 ANTIBODY-GRANULOCYTE REGULATORY FACTOR FUSION PROTEIN, PREPARATION METHOD THEREFOR AND APPLICATION THEREOF

(71) Applicant: SHANGHAI CELGEN BIO-PHARMACEUTICAL CO., LTD., Shanghai (CN)

(72) Inventors: Yi CHEN, Shanghai (CN); Zeling CAI, Shanghai (CN); Peng LI, Shanghai (CN); Yaqin SONG, Shanghai (CN)

(21) Appl. No.: 18/681,283

(22) PCT Filed: Aug. 8, 2022
(86) PCT No.: PCT/CN2022/110926

§ 371 Date: May 23, 2024

(30) Foreign Application Priority Data
Aug. 6, 2021 (CN) 202110902623.8

(51) Int. Cl. C07K 14/535; A61K 38/00; A61P 35/00; C07K 16/32

(52) U.S. Cl. CPC C07K 14/535; A61P 35/00; C07K 16/32; A61K 38/00; C07K 2319/00

(57) ABSTRACT

Provided are an anti-Her-2 antibody-granulocyte regulatory factor fusion protein, a preparation method therefor and an application thereof. Specifically, this relates to a fusion protein, a single chain of the fusion protein comprising the following elements fused together: (a) a first protein element; (b) a second protein element; and (c) optionally, a joint element located between the first protein element and the second protein element. The first protein element is an antigen recognition module, for example, a protein element of an anti-Her-2 antibody or an active fragment thereof; the second protein element is a granulocyte colony stimulating factor. The present fusion protein has advantages in precise recognition, immunotherapy, controllable toxicity, and enhanced ADCC, and also has the synergistic effect of high-efficiency tumor cell killing activity, as well as low toxicity and side effects.

### TECHNICAL FIELD

The present invention relates to the field of biotechnology and medicine, and specifically relates to an anti-Her-2 antibody-granulocyte regulatory factor fusion protein and its preparation method and application.

### BACKGROUND

HER-2 is a proto-oncogene, belonging to the human epidermal growth factor receptor family, which inhibits the apoptosis of cancer cells, promotes their proliferation and invasion by regulating downstream signaling pathways, HER2 amplification or overexpression accounts for about 20%-30% in breast cancer patients, in addition, HER2 is often detected in gastric cancer. Trastuzumab, a representative drug of HER-2 target, has achieved good efficacy in HER-2+ breast cancer and gastric cancer, but the current resistance rate and recurrence rate of breast cancer to trastuzumab are increasing year by year, and its final resistance rate is as high as 65%, including 70% of patients who are sensitive to trastuzumab treatment at the beginning of treatment and eventually develop drug resistance. Therefore, new combinations are urgently needed to achieve effective control of HER-2+ tumors.

Neutrophils are one of the most important defense systems in tissue damage, accounting for about 50% to 70% of circulating leukocytes. Recent studies have shown that tumor-associated neutrophils (TANs) also play an important role in tumor immunity. In addition to direct killing of tumor cells through mechanisms such as granzyme B, neutrophils can also induce apoptosis through antibody-dependent cell-mediated cytotoxicity (ADCC). Therefore, by enhancing the ADCC activity of anti-tumor antibody drugs, the function of neutrophils to kill tumor cells can be improved.

In summary, there is an urgent need to develop a safer, more effective, and precise fusion protein of anti-Her-2 antibody-granulocyte regulatory factor targeting Her-2 high-expressing tumors.

### SUMMARY OF THE INVENTION

The purpose of the present invention is to provide a safer, more effective and precise anti-Her-2 antibody-granulocyte regulatory factor fusion protein targeting tumors.

The purpose of the present invention is to provide an anti-Her-2 antibody-granulocyte regulatory factor fusion protein and its use.

In the first aspect of the present invention, it provides a fusion protein single strand, and the fusion protein single strand comprises the following elements fused together:
(a) first protein element;
(b) secondary protein element; and
(c) optional linker element between the first and second protein elements;
wherein, the first protein element is an antigen recognition module;
the second protein element is granulocyte colony-stimulating factor.

In another preferred embodiment, the antigen recognition module comprises an antibody or an active fragment thereof, and the antibody is selected from the following group: anti-CD20 antibody, anti-TIM-3 antibody, anti-LAG-3 antibody, anti-CD73 antibody, anti-CD47 antibody, anti-DLL3 antibody, anti-FRmAb antibody, anti-CTLA-4 antibody, anti-OX40 antibody, anti-CD137 antibody, anti-PD-1 antibody.

In another preferred embodiment, the antibody is a monoclonal antibody.

In another preferred embodiment, the active fragment is an active fragment comprises F(ab), F(ab′)2, scFv, VH, CH, VL, or VHH of the antibody.

In another preferred embodiment, the antigen recognition module is an anti-Her-2 antibody or an active fragment thereof.

In another preferred embodiment, the anti-Her-2 antibody or its active fragment is an active fragment comprises F(ab), F(ab′)2, scFv, VH, CH, VL, or VHH.

In another preferred embodiment, the anti-Her-2 antibody or its active fragment is selected from the active fragment of trastuzumab.

In another preferred embodiment, the linker element is a peptide bond or a peptide linker.

In another preferred embodiment, the granulocyte colony-stimulating factor (G-CSF) is derived from human or non-human mammals, and preferably from rodents (e.g., mice, rats), primates, and humans.

In another preferred embodiment, the G-CSF comprises wild-type and mutant.

In another preferred embodiment, the G-CSF comprises a full-length, mature form of the G-CSF, or its active fragment.

In another preferred embodiment, the G-CSF also comprises a derivative of the G-CSF.

In another preferred embodiment, the derivatives of the G-CSF comprises modified G-CSF, a protein molecule whose amino acid sequence is homologous to natural G-CSF and has native G-CSF activity, a dimer or multimer of G-CSF, and a fusion protein containing an amino acid sequence of G-CSF.

In another preferred embodiment, the “a protein molecule whose amino acid sequence is homologous to natural G-CSF and has natural G-CSF activity” refers to a protein molecule whose amino acid sequence has ≥85% homology compared with G-CSF, preferably ≥90% homology, more preferably ≥95% homology, most preferably ≥98% homology, and has G-CSF activity.

In the second aspect of the present invention, it provides a fusion protein consist of a single strand of a fusion protein described in the first aspect of the present invention, and the fusion protein comprises two single strands, each of which from the N-terminus to the C-terminus has a structure as shown in the following equation I:

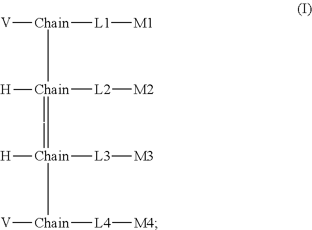

wherein, M1, M2, M3 and M4 were independently none or granulocyte colony-stimulating factor G-CSF, and at least one of them was not none;
L1, L2, L3, and L4 are independently none or bond or peptide linkers;
wherein,

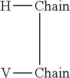

is a protein element of an anti-Her-2 antibody or its active fragment, wherein,
H-Chain is an anti-Her-2 antibody heavy chain or its active fragment;
V-Chain is an anti-Her-2 antibody light chain or its active fragment;
[symbol] denotes the disulfide bond between the heavy and light chains;
“−” denotes the peptide bond.

In another preferred embodiment, the [symbol] is one or more interchain disulfide bonds between heavy or light chains.

In another preferred embodiment, the M1, M4, L1, L2, L3 and L4 are none.

In another preferred embodiment, the M2, M3, L2 and L3 are none.

In another preferred embodiment, the fusion proteins each have a structure selected from the following equations II, III, IV, or V:

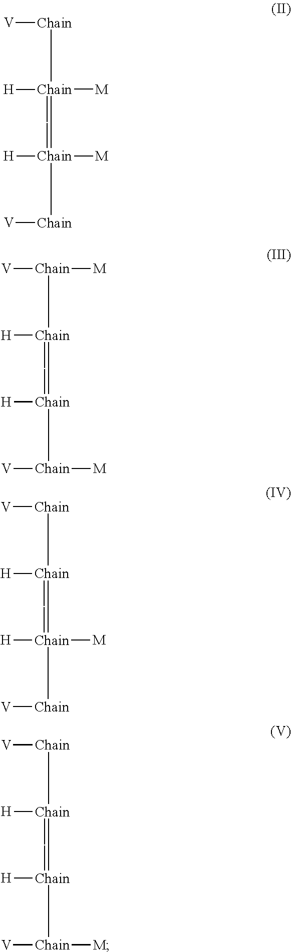

wherein,

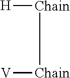

is a protein element of an anti-Her-2 antibody or its active fragment, wherein,
H-Chain is an anti-Her-2 antibody heavy chain or its active fragment;
V-Chain is an anti-Her-2 antibody light chain or its active fragment;
M is granulocyte colony-stimulating factor;
[symbol] denotes the disulfide bond between the heavy and light chains;
“−” denotes the peptide bond or peptide linker.

In another preferred embodiment, the fusion protein is a dimer.

In another preferred embodiment, the fusion protein is homologous or heterodimer.

In another preferred embodiment, the heavy chain active fragment includes or contains the heavy chain, VH, CH, VHH, Fc region, or HCDR of the anti-Her-2 antibody.

In another preferred embodiment, the light chain active fragment includes or contains light chains, VL, CL, or LCDR of an anti-Her-2 antibody.

In another preferred embodiment, the anti-Her-2 antibody heavy chain or its active fragment comprises a heavy chain variable region, a heavy chain constant region, and an Fc segment.

In another preferred embodiment, the Fc fragment is derived from human or non-human mammals, and preferably from rodents (e.g., mice, rats), primates, and humans.

In another preferred embodiment, the Fc fragment is the Fc fragment of the immunoglobulin IgG, preferably the Fc part of IgG1.

In another preferred embodiment, the anti-Her-2 antibody light chain or its active fragment comprises a light chain variable region, a light chain constant region.

In another preferred embodiment, H-Chain is the heavy chain of trastuzumab.

In another preferred embodiment, V-Chain is the light chain of trastuzumab.

In another preferred embodiment, M is granulocyte colony-stimulating factor G-CSF.

In another preferred embodiment, in the fusion protein, the H-Chain or V-Chain is linked to the G-CSF in a head-head, head-tail, or tail-tail manner.

In another preferred embodiment, the “head” refers to the N-terminus of the peptide or its fragment, especially the N-terminus of the wild-type peptide or its fragment.

In another preferred embodiment, the “tail” refers to the C-terminus of the polypeptide or its fragment, especially the C-terminus of the wild-type polypeptide or its fragment.

In another preferred embodiment, the length of the peptide linker is 0-20 amino acids, preferably 1-15 amino acids.

In another preferred embodiment, the H-Chain comprises or has amino acids on positions 1-449 in SEQ ID NO: 11, the V-Chain comprises or has amino acids on positions 1-214 in SEQ ID NO: 14, and the G-CSF contains or has amino acids on positions 450-624 in SEQ ID NO: 11, or amino acids on positions 222-396 in SEQ ID NO: 14.

In another preferred embodiment, the peptide linker sequence is positions 215-221 in SEQ ID NO: 14.

In another preferred embodiment, the sequence of the fusion protein is selected from the following group:
(1) the sequence of H-chain-M as shown in SEQ ID NO:11 and the sequence of V-chain as shown in SEQ ID NO:12;
(2) the sequence of H-chain as shown in SEQ ID NO:13 and the sequence of V-chain-M as shown in SEQ ID NO:14;
(3) derived sequences having ≥80% homology to the above sequences (preferably, ≥90% homology; so preferably ≥95% homology; most preferably, ≥97% homology, such as more than 98%, more than 99%).

In another preferred embodiment, the H-chain-M is a fusion protein heavy chain with a sequence as shown in SEQ ID NO: 11.

In another preferred embodiment, the V-chain-M is a light chain of the fusion protein, with a sequence as shown in SEQ ID NO: 14.

In the third aspect of the present invention, it provides a separated polynucleotide, and the polynucleotide encoding a fusion protein as described in the second aspect of the present invention.

In another preferred embodiment, the polynucleotide also contains additional auxiliary elements selected from the following group on the flanks of the ORF of the mutant or fusion protein: a signal peptide, a secretory peptide, a tag sequence (e.g., 6His), and a combination thereof.

In another preferred embodiment, the polynucleotide is selected from the group consisting of DNA sequence, RNA sequence, and a combination thereof.

In the fourth aspect of the present invention, it provides a vector, which contains the polynucleotide as described in the third aspect of the present invention.

In another preferred embodiment, the vector includes: a bacterial plasmid, a bacteriophage, a yeast plasmid, a plant cell virus, a mammalian cell virus such as an adenovirus, a retrovirus, or other vectors.

In another preferred embodiment, the vector comprises one or more promoters operably linked to the nucleic acid sequence, enhancer, transcription termination signal, polyadenylation sequence, origin of replication, selectable marker, nucleic acid restriction site, and/or homologous recombination site.

In another preferred embodiment, the vector includes an expression vector, a shuttle vector, and an integration vector.

In the fifth aspect of the present invention, it provides a host cell, the host cell contains the vector of the fourth aspect of the present invention, or its genome integrated by the polynucleotide of the third aspect of the present invention.

In another preferred embodiment, the host cells including prokaryotic cells and eukaryotic cells.

In another preferred embodiment, the host cell includes mammalian cells.

In another preferred embodiment, the host cell is an eukaryotic cell, such as yeast cell, plant cell or mammalian cell (including a human and non-human mammal).

In another preferred embodiment, the host cell is a prokaryotic cell, such as Escherichia coli.

In another preferred embodiment, the yeast cell is selected from one or more sources of yeast from the group consisting of: Pichia pastoris, Kluyveromyces and a combination thereof; preferably, the yeast cell includes: Kluyveromyces, more preferably Kluyveromyces marxianus, and/or Kluyveromyces lactis.

In another preferred embodiment, the host cell is selected from the group consisting of Escherichia coli, wheat germ cell, insect cell, SF9, Hela, HEK293, CHO (such as CHOKS), yeast cell, and a combination thereof.

The sixth aspect of the present invention, it provides a method for producing the fusion protein of the second aspect of the present invention, the method comprising the steps of:
(1) under conditions suitable for expression, culturing the host cells as described in the fifth aspect of the present invention, thereby expressing the fusion protein as described in the second aspect of the present invention; and
(2) optionally isolating the fusion protein.

In the seventh aspect of the present invention, it provides a pharmaceutical composition, which comprises:
the fusion protein as described in the second aspect of the present invention, and
pharmaceutically acceptable carriers.

In another preferred embodiment, the drug composition further comprises an additional active ingredient, preferably comprises: a small molecule compound, a cytokine, an antibody (such as an anti-PD-1 antibody, an anti-OX40 antibody, an anti-CD137 antibody, an anti-CD47 antibody, an ADC, a CAR-immune cell).

In another preferred embodiment, the drug composition is in an injectable dosage form.

In the eighth aspect of the present invention, it provides an immune cell, and the immune cell carries a fusion protein as described in the second aspect of the present invention.

In another preferred embodiment, the immune cells comprise T cells.

In the ninth aspect of the present invention, it provides a pharmaceutical composition, and the composition comprises:
the immune cells as described in the eighth aspect of the present invention, and pharmaceutically acceptable carriers.

In the tenth aspect of the present invention, it provides a use of the fusion protein as described in the second aspect of the present invention or the immune cell as described in the eighth aspect of the present invention for the preparing a drug, the drug is used for the treatment of tumors.

In another preferred embodiment, the tumors including breast cancer tumors, gastric cancer tumors, bladder cancer tumors, pancreatic cancer tumors, colorectal cancer tumors, lung cancer tumors, liver cancer tumors, and melanoma tumors.

In another preferred embodiment, the drug used to treat the tumor may be used in combination with another tumor immunotherapy, including but not limited to: chemotherapy, anti-CD20 mAb, anti-TIM-3 mAb, anti-LAG-3 mAb, anti-CD73 mAb, anti-CD47 mAb, anti-DLL3 mAb, anti-FRmAb mAb, anti-CTLA-4 antibody, anti-OX40 antibody, anti-CD137 antibody, anti-PD-1 antibody, PD-1/PD-L1 therapy, other immuno-oncology drugs, anti-angiogenic agents, radiation therapy, antibody-drug conjugates (ADCs), targeted therapies, or other anti-cancer drugs.

The eleventh aspect of the present invention, it provides a method for preventing and/or treating tumors, comprising steps: administering the fusion protein described in the second aspect of the present invention to a subject in need.

In another preferred embodiment, the fusion protein is administered in the form of monomers and/or dimers.

In another preferred embodiment, the subject is a human.

In another preferred embodiment, the tumors including breast cancer tumors, gastric cancer tumors, bladder cancer tumors, pancreatic cancer tumors, colorectal cancer tumors, lung cancer tumors, liver cancer tumors, and melanoma tumors.

It should be understood that, within the scope of the present invention, each technical feature of the present invention described above and in the following (as examples) may be combined with each other to form a new or preferred technical solution, which is not listed here due to space limitations.

### DESCRIPTION OF DRAWINGS

FIG. 1 shows schematic diagrams of four of the structures of the embodiment of the anti-Her-2 antibody-granulocyte colony-stimulating factor fusion protein of the present invention (FIG. 1A, FIG. 1B, FIG. 1C, FIG. 1D).

FIG. 2 shows the SDS-PAGE electrophoresis analysis of the heavy chain-G-CSF trastuzumab fusion protein. FIG. 2A: Non-reduced 6% SDS-PAGE electrophoresis analysis. FIG. 2B: Reduced 10% SDS-PAGE electrophoresis analysis. Lane 1 is trastuzumab, lane 2 is the heavy chain-G-CSF trastuzumab fusion protein, and MW is the protein molecular weight standard (kDa).

FIG. 3 shows an ELISA study of the heavy chain-G-CSF trastuzumab fusion protein binding to recombinant human Her-2 ECD in vitro (FIG. 3A), and a flow cytometry analysis study of membrane Her-2 binding (FIG. 3B).

FIG. 4 shows the study of the inhibition of Her-2-positive breast cancer cells BT-474 growth by the heavy chain-G-CSF trastuzumab fusion protein in vitro.

FIG. 5 shows a study of the stimulation of mouse myeloid leukemia lymphocytes NFS-60 growth by the heavy chain-G-CSF trastuzumab fusion protein in vitro

FIG. 6 shows the fusion protein of heavy chain G-CSF trastuzumab promotes neutrophil proliferation in mouse blood in vivo.

FIG. 7 shows the study of the inhibition of human Her-2-expressing mouse melanoma cell B16 growth by the heavy chain-G-CSF trastuzumab fusion protein in mice. FIG. 7A: Mean tumor volume curve of mice in each group; FIG. 7B: Mean weight curve of mice in each group.

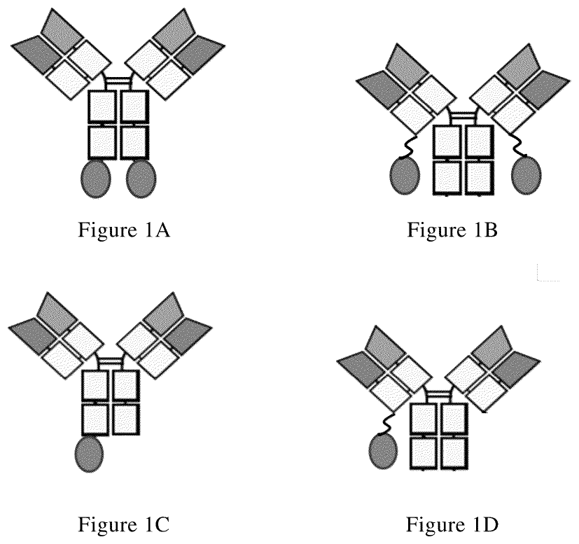

### EMBODIMENT

Through extensive and intensive research, the present inventor accidentally discovered for the first time that the fusion protein obtained by fusing (a) anti-Her-2 antibody or its active fragment and (b) granulocyte colony-stimulating factor G-CSF has a synergistic effect of high efficiency in killing tumor cell activity and low toxicity and side effects. Specifically, the present invention relates to a novel fusion protein consisting of a tumor-related targeting element, a preferred monoclonal antibody or its fragment, and a colony-stimulating factor. It specifically recognizes molecules expressed on human tumors, such as the human epidermal growth factor receptor (HER-2), and carries granulocyte colony-stimulating factor (G-CSF). The obtained fusion protein can specifically bind to HER-2 expressed by tumor tissues, inhibit tumor growth, and deliver G-CSF to targeted tumor tissues, enhance the role of neutrophils in killing tumors. The new fusion protein can be used in the treatment of HER2+ tumors. The present invention has been completed on the basis of this.

The fusion protein according to the present invention is composed of the following two parts: (1) a full-length monoclonal antibody or a minimum antigen-recognizing part of the tumor-specific antigen Her-2, and (2) a cytokine that regulates neutrophil proliferation, differentiation and activation, such as granulocyte colony-stimulating factor G-CSF. Recombinant DNA technology was used to construct a fusion protein encoding a ribonucleotide, which contains an anti-Her-2 antibody heavy chain with or without CH1 or CH2 or CH3 of the heavy chain constant region, and its C-terminus is fused with an active cytokine such as G-CSF. When the fusion protein heavy chain expression plasmid is co-transfected with the anti-Her-2 antibody light chain expression plasmid, the fusion protein of anti-Her-2 antibody-cytokine (e.g., G-CSF) can be generated, which can bind to Her-2-expressing tumor cells and deliver cytokines to the tumor site. Similarly, biologically active neutrophil regulatory cytokines can also be fused with anti-Her-2 single-chain antibodies, and the complete fusion protein is a polypeptide chain, and each functional region is connected by a linker peptide to ensure that the fusion protein has the correct spatial structure and maintains its biological activity.

The fusion proteins of the present invention are a new class of molecules with two biological functions: first, they can target Her-2-expressing tumor tissues and inhibit tumor growth by blocking the function of Her-2, or kill tumor cells through ADCC or CDC functions. Second, they can specifically deliver biologically active cytokines to the tumor site. These cytokines have the function of regulating the activity of immune cell. Therefore, they can increase the infiltration of immune cell tumor tissue and enhance the activity of immune cells, so that the growth of tumors, such as breast cancer, gastric cancer, etc., can be inhibited. Since cytokines are mainly confined to the site of tumor tissue, the toxicity caused to patients is relatively small. Therefore, the purpose of the present invention is to provide an antibody-cytokine fusion protein containing a monoclonal antibody or antibody fragment targeting a tumor expressing Her-2 and fusing with a biologically active cytokine.

The antibody in the fusion protein of the present invention can be a full-length antibody or a key fragment of the antibody, such as scFv, F(ab)2, etc. Theoretically, all antibodies that can bind to the Her-2 receptor on the tumor cell membrane are suitable for the construction of the antibody-granulocyte colony-stimulating factor fusion protein of the present invention. In the present invention, trastuzumab is the preferred antibody.

The purpose of the present invention is to provide an antibody-granulocyte colony-stimulating factor fusion protein in which the antibody part is a full-length antibody or an antibody fragment containing a necessary variable region sequence, such as F(ab) or F(ab)2 or scFv. The fusion protein cytokine part of the present invention is selected from a biologically active G-CSF and is directly or linked to the antibody part by linking the peptide chain.

The content of the invention also comprises a method for producing and preparing an antibody-cytokine fusion protein, by directly or indirectly fusing the nucleotide sequence encoding the antibody and the nucleotide sequence of the cytokine, cloning it onto an expression vector, and then transfecting the vector into the cell, cultivating the transfected cell in a suitable medium, and obtaining an antibody-cytokine fusion protein.

The antibody-cytokine fusion protein of the present invention can be used for clinical tumor treatment. Therefore, the content of the present invention comprises the composition of a clinical therapeutic drug, which contains at least one of the fusion proteins of the present invention and a physiologically acceptable carrier.

### The Terms

Unless otherwise defined, all technical and scientific terms used herein have the same meanings as commonly understood by a person skilled in the art to which the invention belongs.

In the present invention, the terms “fusion protein of the present invention”, “Her-2 antibody-granulocyte colony-stimulating factor fusion protein of the present invention”, and “Her-2 antibody-G-CSF fusion protein” are used interchangeably, all referring to the fusion protein mentioned in the second aspect of the present invention.

As used herein, when used in reference to a specific value, the term “approximately” means that the value can vary by no more than 1% from the listed value. For example, as used in this article, the expression “about 100” includes all values between 99 and 101 (e.g., 99.1, 99.2, 99.3, 99.4, etc.).

As used herein, Fc refers to the Fc fragment of human immunoglobulin, unless otherwise stated. The term “immunoglobulin Fc region” refers to the immunoglobulin chain constant region, specifically the carboxyl terminus of or part thereof of the immunoglobulin heavy chain constant region, e.g. the immunoglobulin Fc region may including a combination of two or more domains of the heavy chains CH1, CH2, CH3 and the immunoglobulin hinge region, preferably the used immunoglobulin Fc region including at least a immunoglobulin hinge region, a CH2 domain and a CH3 domain, preferably absence the CH1 domain.

It is known that there are many classes of human immunoglobulins, such as IgA, IgD, IgE, IgM, and IgG (including four subclasses: IgG1, IgG2, IgG3, and IgG4), selecting specific immunoglobulin Fc regions from specific immunoglobulin categories and subclasses is within the scope of expertise of those skilled in the art. In a preferred example, the immunoglobulin Fc region may optionally contain the coding sequence of the Fc region of the human immunoglobulin IgG4 subclass, in which one immunoglobulin heavy chain 1 domain (CH1) is missing, but the hinge region and the coding sequences of CH2, CH3, and two domains are included.

As used herein, the “contain”, “have” or “include” include “contains”, “mainly consists of . . . composition”, “basically composed of . . . composed of”, and composed of . . . composition”; “mainly caused by . . . composition”, “basically composed of . . . the composition of” and “consists of . . . constitutes” belonging to the subordinate concept of “having” or “including”.

### Neutrophils

Neutrophils are one of the most important defense systems in tissue damage, accounting for about 50% to 70% of circulating leukocytes. Recent studies have shown that tumor-associated neutrophils (TANs) also play an important role in tumor immunity. Under the induction of some cytokines, neutrophils can achieve effective killing of tumor cells, such as under the induction of TNFα, neutrophils kill tumor cells through the ROS pathway, and under the induction of IFN-γ and IL-2, neutrophils directly toxicity to tumor cells by expressing granzyme B. Neutrophils can also induce apoptosis of tumor cells through antibody-dependent cell-mediated cytotoxicity (ADCC). Under the induction of radiotherapy, it will cause a large number of neutrophils to infiltrate tumor tissues, and finally induce apoptosis of tumor cells through the ROS pathway. In addition, neutrophils can express necrosis factor-related apoptosis ligand (TRAIL) and myeloperoxidase (MPO) with direct killing activity to exert anti-tumor effects. In addition to the above-mentioned direct killing effects, neutrophils can also indirectly enhance antitumor effects by stimulating T cell proliferation, promoting IFN-γ release, and activating dendritic cells. Neutrophils are mainly regulated by granulocyte colony-stimulating factor (G-CSF), which induces neutrophil proliferation, differentiation, and release to the peripheral blood. A study showed that neutrophils involved in anti-tumor depletion occurred after a certain period of time due to their short half-life, and significant tumor growth was observed after neutrophil depletion. If G-CSF is added at the same time as treatment to promote the activation and migration of neutrophils in the tumor microenvironment, the number of neutrophils will increase significantly, the tumor immune response will be significantly enhanced, and the growth of the tumor will be effectively inhibited.

### Fusion Proteins

As used herein, unless otherwise noted, the fusion protein is an isolated protein that is not associated with other proteins, peptides, or molecules, and is expressed by recombinant host cells, or is the product of isolation or purification.

The fusion protein constructed by the present invention is composed of the following two parts:
(1) a full-length monoclonal antibody or a minimum antigen-recognizing part of the tumor-specific antigen Her-2;
(2) a biologically active granulocyte colony-stimulating factor in the granulocyte colony-stimulating factor family, such as G-CSF or GM-CSF.

Recombinant DNA technology was used to construct a fusion protein encoding a ribonucleotide, which contains an anti-Her-2 antibody heavy chain with or without CH1 or CH2 or CH3 of the heavy chain constant region, and its C-terminus is fused with an active granulocyte colony-stimulating factor.

When the fusion protein heavy chain expression plasmid is co-transfected with the anti-Her-2 antibody light chain expression plasmid, an anti-Her-2 antibody-granulocyte colony-stimulating factor (e.g., G-CSF) fusion protein can be generated, which can bind to Her-2-expressing tumor cells and deliver granulocyte colony-stimulating factor to the tumor site.

The bioactive granulocyte colony-stimulating factor is fused with a single-chain antibody anti-Her-2, and the complete fusion protein is a polypeptide chain, and each functional region is connected by a linker peptide to ensure that the fusion protein has the correct spatial structure and maintains its biological activity.

The fusion proteins of the present invention are a new class of molecules with two biological functions: first, they can target Her-2-expressing tumor tissues, and second, they can specifically deliver biologically active cytokines to the tumor site. These cytokines have the function of regulating the activity of immune cells, therefore, they can increase the infiltration of immune cell tumor tissue and enhance the activity of immune cells, so that the growth of tumors, such as breast cancer, gastric cancer, etc., can be inhibited. For granulocyte colony-stimulating factors are mainly confined to tumor tissue sites, the toxicity to patients is relatively small.

The antibody in the fusion protein of the present invention can be a full-length antibody or a key fragment of the antibody, such as scFv, F(ab) 2, or VHH, etc. Theoretically, all antibodies that can bind to the Her-2 receptor on the tumor cell membrane are suitable for the construction of the antibody-granulocyte colony-stimulating factor fusion protein of the present invention (trastuzumab, lapatinizumab, and pertuzumab). In the present invention, trastuzumab is preferred.

The fusion protein granulocyte colony-stimulating factor moiety portion of the present invention is directly or linked to the antibody moiety by a peptide linker.

The present invention provides a fusion protein comprising the following elements:
(a) protein elements of the anti-Her-2 antibody or its active fragment, (b) protein elements of granulocyte colony-stimulating factor (G-CSF), and (c) linker elements. In the fusion protein of the present invention, between the elements (such as between element a and element b), may contain or not contain linkers.

The fusion protein of the present invention not only has a longer in vivo half-life, but can more effectively inhibit the concentration of immune disease-related antibodies (especially IgE) in serum.

According to the amino acid sequence of the present invention, those skilled in the art can conveniently prepare the fusion protein of the present invention by various known methods. These methods including, but are not limited to, recombinant DNA methods, artificial synthesis, etc.

After knowing the amino acid sequence of the fusion protein of the present invention, those skilled in the art can conveniently obtain a gene sequence encoding the fusion protein of the present invention according to the amino acid sequence.

A preferred fusion protein is trastuzumab HC-G-CSF fusion protein, with a heavy nucleotide sequence as shown in SEQ ID NO: 6 and a heavy chain amino acid sequence as shown in SEQ ID NO: 11, where positions 1-449 of the heavy chain amino acid (SEQ ID NO: 11) sequence are the amino acid sequence of trastuzumab, and positions 450-624 are the G-CSF amino acid sequence.

A preferred fusion protein is trastuzumab LC-LINKER-G-CSF fusion protein, with a light chain nucleotide sequence as shown in SEQ ID NO: 9 and a light chain amino acid sequence as shown in SEQ ID NO: 14, where positions 1-214 of the light chain amino acid (SEQ ID NO: 14) sequence are the light chain amino acid sequence of trastuzumab, and positions 222-396 are the G-CSF amino acid sequence.

In another preferred embodiment, the light chain nucleotide sequence of the trastuzumab HC-G-CSF fusion protein of the present invention as shown in SEQ ID NO: 7, and the light chain amino acid sequence as shown in SEQ ID NO: 12.

In another preferred embodiment, the heavy chain nucleotide sequence of trastuzumab LC-G-CSF or LC-linker-G-CSF fusion protein of the present invention as shown in SEQ ID NO: 8, and the heavy chain amino acid sequence as shown in SEQ ID NO: 13.

As used herein, “isolated” means the separation of a substance from its original environment (in the case of natural substances, the original environment is the natural environment). For example, the polynucleotides and peptides in the natural state in living cells are not separated and purified, but the same polynucleotides or peptides are separated and purified if they are separated from other substances present in the natural state.

As used herein, “isolated recombinant fusion protein” means that the recombinant fusion protein is substantially free of other proteins, lipids, sugars, or other substances that are naturally associated with it. A person skilled in the art can purify recombinant fusion proteins using standard protein purification techniques. Essentially pure proteins produce a single main band on non-reducing polyacrylamide gels.

As used herein, the term “fusion protein” also includes variant forms of fusion proteins (such as the sequences shown in SEQ ID NO.: 1 or 2) having the above-mentioned activities. These variants include (but are not limited to): deletions, insertions and/or substitutions of 1-3 (usually 1-2, more preferably 1) amino acids, and additions or deletions of one or several (usually within 3, preferably within 2, more preferably within 1) amino acids at the C-terminus and/or N-terminus. For example, in the art, substitutions with amino acids of close or similar properties generally do not alter the function of the protein. For another example, addition or deletion of one or several amino acids at the C-terminus and/or N-terminus usually does not alter the structure and function of the protein. Furthermore, the term also includes monomeric and multimeric forms of the polypeptides of the invention. The term also includes linear as well as nonlinear polypeptides (e.g., cyclic peptides).

The present invention also includes active fragments, derivatives and analogs of the above fusion proteins. As used herein, the terms “fragment”, “derivative” and “analog” refer to polypeptides that substantially retain the function or activity of the fusion proteins of the present invention. The polypeptide fragments, derivatives or analogs of the present invention may be (i) a polypeptide in which one or several conservative or non-conservative amino acid residues (preferably conservative amino acid residues) have been substituted, or (ii) a polypeptide having a substituent group in one or more amino acid residues, or (iii) a polypeptide formed by fusion of an antigenic peptide with another compound (such as a compound that prolongs the half-life of a polypeptide, such as polyethylene glycol), or (iv) a polypeptide formed by fusing an additional amino acid sequence with this polypeptide sequence (a fusion protein formed by fusing with a leader sequence, a secretory sequence, or a tag sequence such as 6×His). These fragments, derivatives and analogs are well known to those skilled in the art in light of the teachings herein.

A class of preferred active derivatives refers to that compared with the amino acid sequence of Formula I or Formula II, at most 3, preferably at most 2, more preferably at most 1 amino acid is replaced by amino acids with close or similar properties to form a polypeptide. These conservatively variant polypeptides are best produced by amino acid substitutions according to Table A.

TABLE A

| initial residue | representative substitution | Preferred substitution |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The present invention also provides analogs of the fusion proteins of the present invention. The differences between these analogs and the polypeptides shown in SEQ ID NO.: 11, 12, 13 or 14 may be differences in amino acid sequence, differences in modified forms that do not affect the sequence, or both. Analogs also include analogs with residues other than natural L-amino acids (e.g., D-amino acids), as well as analogs with non-naturally occurring or synthetic amino acids (e.g., beta, gamma-amino acids). It should be understood that the polypeptides of the present invention are not limited to the representative polypeptides as exemplified above.

Modified (generally without altering the primary structure) forms include chemically derivatized forms such as acetylation or carboxylation of the polypeptide in vivo or in vitro. Modifications also include glycosylation, such as those resulting from glycosylation modifications in the synthesis and processing of the polypeptide or in further processing steps. Such modifications can be accomplished by exposing the polypeptide to enzymes that perform glycosylation, such as mammalian glycosylases or deglycosylases. Modified forms also include sequences with phosphorylated amino acid residues (e.g., phosphotyrosine, phosphoserine, phosphothreonine). Also included are polypeptides that have been modified to increase their resistance to proteolysis or to optimize their solubility properties.

### Polynucleotides

The polynucleotide of the present invention can be in a form of DNA or RNA. DNA forms include cDNA, genomic DNA, or synthetic DNA. DNA can be single stranded or double stranded. DNA can be the coding strand or the non-coding strand.

The present invention also relates to variants of the above polynucleotides encoding protein fragments, analogs and derivatives of the same amino acid sequence as the present invention. The variants of this polynucleotide may be naturally occurring allelic variants or non-naturally occurring variants. These nucleotide variants include substituted variants, deletion variants, and insertion variants. As is known in the art, an allelic variant is a replacement form of a polynucleotide, which may be a substitution, deletion, or insertion of one or more nucleotides, but does not substantially change its function of encoding a polypeptide.

As used herein, the term “primer” refers to a general name for a oligonucleotide that, when paired with a template, can be used as a starting point for the synthesis of a DNA strand complementary to the template under the action of DNA polymerase. The primer may be a natural RNA, DNA, or any form of natural nucleotide. The primer may even be a non-natural nucleotide such as LNA or ZNA. The primer “roughly” (or “substantially”) is complementary to a particular sequence on a chain on the template. The primer must be fully complementary to a chain on the template to begin extending, but the sequence of the primer does not have to be completely complementary to the sequence of the template. For example, at the 5′ end of the primer complementary to the template at one 3′ end plus a sequence that is not complementary to the template, such primers are still substantially complementary to the template. As long as enough long primers can be fully combined with the template, the incomplete complementary primers can also form a primer-template complex with the template, thereby performing amplification.

According to the amino acid sequence provided by the present invention, the skilled in the art can conveniently prepare the fusion protein of the present invention by using various known methods. These methods are, for example, but are not limited to, recombinant DNA methods, artificial synthesis, etc.

The nucleotide full-length sequence of the fusion protein element (such as an anti-Her-2antibody active fragment or G-CSF) or a fragment thereof can generally be obtained by a PCR amplification method, a recombination method or an artificial synthesis method. For PCR amplification, primers can be designed according to the disclosed related nucleotide sequences, especially open reading frame sequences, and related sequences can be obtained by using a commercially available cDNA library or a cDNA library prepared according to conventional methods known to a person skilled in the art as a template. When the sequence is long, two or more PCR amplification often needs to be performed, and then the amplified fragments are spliced together in the correct order.

Once a relevant sequence is obtained, recombination methods can be used to obtain the relevant sequence in large quantities. This is usually carried out by cloning the sequence into a vector, transforming a cell with the vector, and then separating the relevant sequence from the proliferated host cell by conventional methods.

In addition, a relevant sequence can be synthesized artificially, especially when the fragment is short in length. Usually, several small fragments are synthesized first, and then are linked together to obtain a fragment with a long sequence.

A method of amplification of DNA/RNA using PCR technology is preferred for obtaining genes of the present invention. The primers for PCR can be appropriately selected according to the sequence information of the present invention disclosed herein, and can be synthesized by a conventional method. The amplified DNA/RNA fragment can be isolated and purified by conventional methods such as by gel electrophoresis.

### Vector and Host Cells

The present invention also includes a vector containing the polynucleotide of the present invention, and a host cell engineered by the vector or the coding sequence of the fusion protein of the present invention, and a method for producing the protein by recombination technology.

With the conventional recombinant DNA technique, the polynucleotide sequence of the present invention can be used to express or produce the recombinant protein of the present invention. Generally, the method comprises the following steps:
(1) transforming or transfecting a suitable host cell with a polynucleotide or variant thereof encoding the protein of the present invention or a recombinant expression vector containing said polynucleotide;
(2) culturing the host cell in a suitable culture medium;
(3) isolating and purifying protein from the culture medium or cell.

Method well known to the skilled in the art can be used to construct the expression vector, which contains the DNA sequence coding the protein of the invention and suitable transcription/translation control signals. These methods comprise DNA recombinant technology in vitro, DNA synthesis technology, recombinant technology in vivo, and the like. The DNA sequence can be operably linked to an appropriate promoter in an expression vector to direct mRNA synthesis. The expression vector also comprises a ribosome binding site for translation initiation and a transcription terminator.

Furthermore, the expression vector preferably comprises one or more selectable marker genes to provide phenotypic traits for selection of transformed host cells, such as dihydrofolate reductase, neomycin resistance, and green fluorescent protein (GFP) for eukaryotic cell culture, or tetracycline or ampicillin resistance for Escherichia coli.

A vector comprising the appropriate DNA sequence and the appropriate promoter or control sequence described above may be used to transform an appropriate host cell to enable it to express a protein.

The host cell can be a prokaryotic cell, such as a bacterial cell; or a lower eukaryotic cell, such as a yeast cell; or a higher eukaryotic cell, such as a mammalian cell. Representative examples are: Escherichia coli, bacterial cells of Streptomyces; fungal cells such as yeast; plant cells; insect cells of Drosophila S2 or SF9; CHO, COS, or 293 cells of animal cells, etc.

One particularly preferred cell is a cell of human and non-human mammals, particularly immune cells, including T cells, NK cells.

Transformation of host cells with recombinant DNA can be carried out using conventional techniques well known to the skilled in the art. When the host is a prokaryotic organism such as Escherichia coli, competent cells capable of absorbing DNA can be harvested after an exponential growth period and processed with a CaCl₂) method, and the steps used are well known in the art. Another method is to use MgCl₂. If necessary, the transformation can also be carried out by electroporation. When the host is a eukaryote, the following DNA transfection methods can be used: calcium phosphate coprecipitation method, conventional mechanical method such as micro-injection, electroporation, liposome packaging, etc.

The obtained transformant can be cultured by conventional methods to express the polypeptide encoded by the gene of the present invention. Depending on the host cell used, the medium used in the culture may be selected from a variety of conventional medium. Culture is carried out under conditions suitable for host cell growth. After the host cell has grown to an appropriate cell density, the selected promoter is induced by a suitable method (such as temperature conversion or chemical induction) and the cells are cultured for a further period of time.

The protein in the above method may be expressed in the cell, or on the cell membrane, or secreted outside the cell. If desired, the physical, chemical and other properties can be utilized in various isolation methods to isolate and purify protein. These methods are well-known to those skilled in the art Examples of these methods include, but are not limited to, conventional renaturation treatment, treatment by protein precipitant (salt precipitation), centrifugation, cell lysis by osmosis, sonication, supercentrifugation, molecular sieve chromatography (gel chromatography), adsorption chromatography, ion exchange chromatography, high performance liquid chromatography (HPLC), and any other liquid chromatography, and the combination thereof.

### Peptide Linkers

The bifunctional fusion protein of the present invention may optionally with or without a peptide linker. Peptide linker size and complexity may affect protein activity. In general, the peptide linker should be of sufficient length and flexibility to ensure that the two proteins connected have enough degrees of freedom in space to exert their functions. At the same time, the influence of the formation of α helix or β sheet in the peptide linker on the stability of the fusion protein is avoided.

The length of the linker peptide is generally 0-20 amino acids, preferably 1-15 amino acids.

Examples of preferred peptide linkers including (but are not limited to): GSGGGGS, (GGGGS)ₙ, wherein n is an integer from 1-8 and n is preferably 1, 2, or 3.

In a specific embodiment of the present invention, the amino acid sequence of the peptide linker is positions 215-221 in the trastuzumab LC-Linker-CCL11 amino acid sequence (SEQ ID NO: 14).

### Pharmaceutical Composition and Methods of Administration

The present invention further provides a composition, comprising (a) an effective amount of the fusion protein of the present invention or an effective amount of the immune cell of the present invention, and a pharmaceutically acceptable carrier.

Typically, the fusion protein of the present invention may be formulated in a non-toxic, inert and pharmaceutically acceptable aqueous carrier medium, wherein the pH is typically about 5-8 and preferably about 6-8.

As used herein, the term “effective amount” or “effective dose” refers to an amount that can be functional or active to humans and/or animals and that can be accepted by humans and/or animals, such as 0.001-99 wt %; preferably 0.01-95 wt %; more preferably, 0.1-90 wt %.

When the pharmaceutical composition of the present invention contains immune cells, an “effective amount” or “effective dose” refers to 1×10³ to 1×10⁷ of the immune cells/ml.

As used herein, a “pharmaceutically acceptable” component is a substance suitable for humans and/or mammals without excessive adverse side reactions (such as toxicity, stimulation, and allergy), i.e. having a reasonable benefit/risk ratio. The term “pharmaceutically acceptable carrier” refers to the carrier for using in administering the therapeutic agents, including various excipients and diluents.

The pharmaceutical composition of the present invention contains a safe and effective amount of the fusion protein of the present invention and a pharmaceutically acceptable carrier. Such carriers include, but are not limited to, saline, buffer solution, glucose, water, glycerin, ethanol or the combination thereof. In general, the pharmaceutical preparation should be matched with the administration mode, and the pharmaceutical composition of the present invention can be prepared in the form of injection, for example, prepared by a conventional method using physiological saline or an aqueous solution containing glucose and other adjuvants. The pharmaceutical composition is preferably manufactured under sterile conditions. The dosage of the active ingredient is a therapeutically effective amount. The pharmaceutical formulation of the present invention may also be made into a sustained release formulation.

The effective amount of the fusion protein of the present invention may vary with the administration mode and the severity of the disease to be treated. The selection of a preferred effective amount may be determined by one of ordinary skill in the art according to various factors (e.g., by clinical trials). The factors include, but are not limited to, the pharmacokinetic parameters of the fusion protein of the present invention, such as bioavailability, metabolism, half-life period, etc.; the severity of the disease to be treated by the patient, the weight of the patient, the immune condition of the patient, the route of administration, and the like. Generally, when the fusion protein of the present invention is administered daily at a dose of about 5 mg-20 mg/kg animal weight (preferably 5 mg-10 mg/kg animal weight), a satisfactory effect can be achieved. For example, a number of separate doses may be given every day, or a dose may be proportionally reduced, by an urgent need for treatment conditions.

The fusion protein of the present invention is particularly suitable for treating diseases such as tumors. Representative tumors include (but are not limited to): breast cancer tumors, gastric cancer tumors, bladder cancer tumors, pancreatic cancer tumors, colorectal cancer tumors, lung cancer tumors, liver cancer tumors, melanin tumors.

The main advantages of the present invention include:
(1) The present invention constructs a new combination of anti-Her-2 antibody and G-CSF, which targets tumor tissues expressing Her-2 and inhibits tumor growth by blocking the function of Her-2, or kills tumor cells by ADCC or CDC function. It has the advantages of accurate identification, immunotherapy, controllable toxicity, and ADCC enhancement.
(2) The fusion protein of the present invention can specifically deliver biologically active cytokines to the tumor site. These cytokines have the function of regulating the activity of immune cells, therefore, they can increase the infiltration of immune cell tumor tissue and enhance the activity of immune cells, so that the growth of tumors, such as breast cancer, gastric cancer, etc., can be inhibited. Since cytokines are mainly confined to the site of tumor tissue, the toxicity caused to patients is relatively small.

The present invention will be further illustrated below with reference to the specific examples. It is to be understood that these examples are for illustrative purposes only and are not intended to limit the scope of the invention. For the experimental methods in the following examples, in which the specific conditions are not specifically indicated, they are performed under routine conditions, e.g., those described by Sambrook. et al., in Molecule Clone: A Laboratory Manual, New York: Cold Spring Harbor Laboratory Press, 1989, or as instructed by the manufacturers, unless otherwise specified. Unless indicated otherwise, parts and percentage are weight parts and weight percentage.

The sequence of the fusion protein of the present invention

| SEQ | | |
|---|---|---|
| ID | | |
| NO. | name | sequence |
| 11 | Trastuzumab | EVQLVESGGGLVQPGGSLRLSCAASGFNIKDTYIHWVRQAPGKGLEWV |
| | fusion protein | ARIYPTNGYTRYADSVKGRFTISADTSKNTAYLQMNSLRAEDTAVYYC |
| | heavy | SRWGGDGFYAMDYWGQGTLVTVSSASTKGPSVFPLAPSSKSTSGGTA |
| | chain-G-CSF | ALGCLVKDYFPEPVTVSWNSGALTSGVHTFPAVLQSSGLYSLSSVVTV |
| | amino acid | PSSSLGTQTYICNVNHKPSNTKVDKKVEPKSCDKTHTCPPCPAPELLGG |
| | sequence | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGATPLGPASSLPQSFLLKCLEQVRKIQGDGAAL |
| | | QEKLCATYKLCHPEELVLLGHSLGIPWAPLSSCPSQALQLAGCLSQLHS |
| | | GLFLYQGLLQALEGISPELGPTLDTLQLDVADFATTIWQQMEELGMAP |
| | | ALQPTQGAMPAFASAFQRRAGGVLVASHLQSFLEVSYRVLRHLAQP |
| 12 | Light chain | DIQMTQSPSSLSASVGDRVTITCRASQDVNTAVAWYQQKPGKAPKLLI |
| | amino acid | YSASFLYSGVPSRFSGSRSGTDFTLTISSLQPEDFATYYCQQHYTTPPTF |
| | sequence of | GQGTKVEIKRTVAAPSVFIFPPSDEQLKSGTASVVCLLNNFYPREAKVQ |
| | trastuzumab | WKVDNALQSGNSQESVTEQDSKDSTYSLSSTLTLSKADYEKHKVYACE |
| | fusion | VTHQGLSSPVTKSFNRGEC |
| | protein | |
| 13 | heavy | EVQLVESGGGLVQPGGSLRLSCAASGFNIKDTYIHWVRQAPGKGLEWV |
| | chain | ARIYPTNGYTRYADSVKGRFTISADTSKNTAYLQMNSLRAEDTAVYYC |
| | amino acid | SRWGGDGFYAMDYWGQGTLVTVSSASTKGPSVFPLAPSSKSTSGGTA |
| | sequence of | ALGCLVKDYFPEPVTVSWNSGALTSGVHTFPAVLQSSGLYSLSSVVTV |
| | trastuzumab | PSSSLGTQTYICNVNHKPSNTKVDKKVEPKSCDKTHTCPPCPAPELLGG |
| | fusion | PSVFLFPPKPKDTLMISRTPEVTCVVVDVSHEDPEVKFNWYVDGVEVH |
| | protein | NAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 14 | Trastuzumab | DIQMTQSPSSLSASVGDRVTITCRASQDVNTAVAWYQQKPGKAPKLLI |
| | fusion protein | YSASFLYSGVPSRFSGSRSGTDFTLTISSLQPEDFATYYCQQHYTTPPTF |
| | light | GQGTKVEIKRTVAAPSVFIFPPSDEQLKSGTASVVCLLNNFYPREAKVQ |
| | chain-linker- | WKVDNALQSGNSQESVTEQDSKDSTYSLSSTLTLSKADYEKHKVYACE |
| | G-CSF | VTHQGLSSPVTKSFNRGECGSGGGGSATPLGPASSLPQSFLLKCLEQVR |
| | amino acid | KIQGDGAALQEKLCATYKLCHPEELVLLGHSLGIPWAPLSSCPSQALQL |
| | sequence | AGCLSQLHSGLFLYQGLLQALEGISPELGPTLDTLQLDVADFATTIWQQ |
| | | MEELGMAPALQPTQGAMPAFASAFQRRAGGVLVASHLQSFLEVSYRV |
| | | LRHLAQP |

### Example 1. Construction of Antibody-G-CSF Fusion Protein Expression Plasmid

Trastuzumab is used as an example of a Her-2 antibody. The intact cDNA encoding the trastuzumab heavy and light chains was synthesized by GenScrip (USA) and cloned on the pUC57 vector, respectively. The cDNA of human G-CSF was purchased from OpenBiosystems (USA).

### 1. Construction of Trastuzumab Heavy Chain-G-CSF Expression Plasmid

There are a large number of reports that during the expression and preparation of monoclonal antibodies, the heavy chain C-terminal lysine of most antibodies is degraded, so when constructing the antibody heavy chain-G-CSF fusion protein, this lysine was removed to maintain the integrity of the antibody-cytokine fusion protein.

The gene encoding the trastuzumab heavy chain was linked to the gene encoding G-CSF by two-step polymerase chain reaction (PCR) method. In the first step, the heavy-chain genes were amplified by PCR method (high-fidelity polymerase Pfx, Invitrogen) using synthetic antibody heavy chain DNA as substrate:
5′ primer M13-F (SEQ ID NO: 1): 5′-TGTAAAACGACGGCCAGT-3′ on the pUC57 vector.
3′ primer KDP004 (SEQ ID NO: 2): 5′-TCCTGGGGACAGTGACAGTG-3′, is a specific primer for antibody heavy chain genes.

Similarly, amplify the gene of the mature G-CSF protein moiety (Ala30-Pro207) by PCR:

5′ primer KDP047 (SEQ ID NO: 3):
5′-CACTGTCACTGTCCCCAGGAGCCACCCCCCTGGGCCC-3′;
3′ primer BGH-R (SEQ ID NO: 4):

5′-AACTAGAAGGCACAGTCGAGGC-3′, on substrate plasmid vector.

The first 20 nucleotide sequences of primer KDP047 are complementary to those of primer KDP004, so that the two PCR fragments can be linked together during the overlapping extension PCR process in the second step.

After the above 2 PCR fragments were purified by DNA glue (Tiangen Biochemical Technology Co., Ltd., Beijing), the second step of overlapping PCR was performed. 5′ primer M13-F (SEQ ID NO: 1), 3′ primer KDP048 (SEQ ID NO: 5), 5′-TGGTGGTGTCTAGAGACTCAGGGCTGGGCAAGGTGG-3′ containing Xba I digestion sequence for cloning.

Trastuzumab heavy chain gene has a Not I cleavage site ahead of the transcription start site, so that after the fragment obtained by overlapping PCR which purified by gel, the Not I/Xba I double digestion (Takara) is performed. Then the digested PCR fragment was cloned into the same digested mammalian cell expression vector. The mammalian cell expression vector was an improved pcDNA3.1 (Invitrogen), the anti-neomycin gene in pcDNA3.1 was replaced by the rat glutamine synthetase gene, and the improved vector was suitable for screening mammalian cells with high expression of stably transfected protein. The recombinant plasmid was transfected into DH5a competent bacteria, and the positive colonies containing the correct recombinant plasmid were identified by colony PCR, and the recombinant plasmid was purified. After digestion and sequencing, trastuzumab heavy chain-G-CSF recombinant gene was identified as having the correct sequence.

### 2. Construction of Trastuzumab Light Chain-G-CSF Expression Plasmid

The gene encoding trastuzumab light chain-linker-G-CSF was synthesized by Suzhou Jinweizhi Biotechnology Co., Ltd. (China) and cloned into the improved pcDNA3.1 vector described above.

### 3. Construction of Trastuzumab Heavy and Light Chain Expression Plasmids

The trastuzumab heavy chain and light chain expression genes in the pUC57 vector were subcloned into the improved pcDNA3.1 vector. The cloning enzymes were Not I and Xba I.

### Example 2. Construction of a Cell Line that Stably Expressing Antibody-G-CSF Fusion Protein

The host cells used for stable expression of these antibody-cytokine fusion proteins were Chinese hamster ovary cells CHO-KS. CHO-KS was CHO-K1 cells grown in fetal bovine serum (FBS)-containing medium, cultured with progressively decreasing FBS content in medium until FBS-free medium culture, and finally acclimated into cells grown in suspension in FBS-free OptiCHO medium (Invitrogen). The anti-neomycin gene of the pcDNA3.1 vector containing the antibody-cytokine fusion protein gene was replaced by the rat glutamine synthetase gene, and the heavy chain and light chain expression plasmids were co-transfected into CHO-KS cells by electroporation (Bio-Rad, Gene Pulser Xcell), and the transfected cells were screened and cultured on 96-well culture plates by limiting dilution method after 24-48 hours of culture. The screening medium was OptiCHO, with 5 μg/ml recombinant human insulin and 10 μM aminosulfoxide methionine (MSX). Cells were cultured in an incubator at 37° C., 8% CO₂. After 3 weeks, the cell culture medium of each well with a cell population was analyzed by ELISA method (alkaline phosphatase-conjugated goat anti-human IgG Fc antibody, Jackson ImmunoResearch Lab), and the cell population with positive antibody-cytokine fusion protein expression was further expanded, and then detected by ELISA, and then expanded, finally a cell line that stably expressing antibody-cytokine fusion protein was obtained.

### Example 3. Preparation and Identification of Trastuzumab-G-CSF Fusion Proteins

The cell lines with high expression of heavy chain-G-CSF trastuzumab and light chain-G-CSF trastuzumab obtained from example 2 were cultured and expanded. Centrifuged the cell culture medium, collected the supernatant, and purified the fusion protein from the supernatant by Protein-A affinity chromatography column.

### Results & Analysis

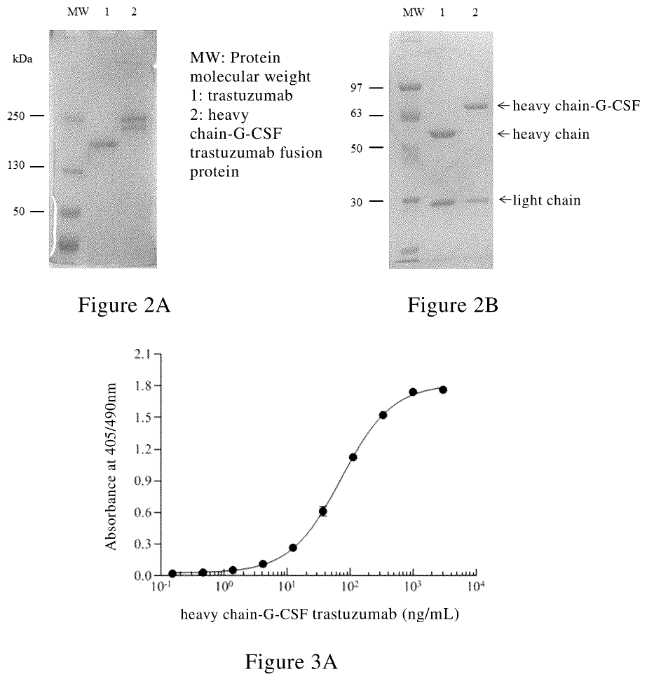

The non-reducing SDS-PAGE gel in FIG. 2A shows that the molecular weight of the heavy chain-G-CSF trastuzumab fusion protein is greater than that of trastuzumab, which is close to its theoretical value of 183 kDa. The reduced SDS-PAGE gel in FIG. 2B shows that the heavy chain-G-CSF trastuzumab fusion protein has a larger molecular weight than the trastuzumab heavy chain, which is consistent with its theoretical molecular weight (68 kDa). The light chain of the heavy chain-G-CSF trastuzumab fusion protein is the same as that of trastuzumab.

### Example 4. Study of Binding of Trastuzumab-G-CSF Fusion Protein to her-2 In Vitro

The binding activity of heavy chain-G-CSF trastuzumab to recombinant human Her-2 extracellular segment (ECD) in vitro was investigated by ELISA.

Recombinant human Her-2 ECD (Sino Biological Technology Co., Ltd.) was dissolved in PBS (pH 7.4) to final concentration of 0.8 μg/mL, and then 50 μL/well was added to a 96-well ELISA plate overnight in a 4° C. freezer. The next day, after 3 washes of the ELISA plates with PBST (PBS with 0.05% Tween-20), 100 μL/well PBST with 3% BSA blocking solution was added and placed in a 37° C. incubator for 1 hour. Heavy chain-G-CSF trastuzumab and trastuzumab were diluted in PBST containing 1% BSA binding solution to prepare a 3-fold serial dilution solution. The blocking solution was discarded and added the diluted antibodies, respectively, 50 μL/well, and reacted in 37° C. incubator for 1 hour. The solution was discarded. The ELISA plates were washed 3 times with PBST, 50 μL/well of secondary antibody (alkaline phosphatase-conjugated goat anti-human IgG Fab antibody, Jackson ImmunoResearch Lab) was added, and reacted in 37° C. incubator for 1 hour. The chromogenic antibody was discarded and the ELISA plate was added 200 μL/well PBST wash solution, placed the ELISA plate on a horizontal shaker for 5 minutes at 100 rpm, the wash solution was discarded, and repeated the wash 4 times. After added 50 μL/well of antibody chromogenic solution (PNPP), the ELISA plate was placed in a 37° C. incubator for color development. The plate was read at a wavelength of 405 nm/490 nm by microplate reader.

The binding of heavy chain-G-CSF trastuzumab to Her-2 protein on cell membranes was studied by flow cytometry. Human breast cancer cells BT-474 (purchased from the Cell Bank of the Chinese Academy of Sciences) is a Her-2 high-expression cell line. An appropriate number of BT-474 cells were taken and adjusted cell density to 2×10⁶/ml with pre-chilled FACS working solution (PBS containing 0.1% FBS), aliquoted 100 μL/tube, blocked on ice for 1 h. Then, trastuzumab-G-CSF fusion protein and trastuzumab were diluted to 100 μg/mL with FACS solution, and then serially diluted. And 10 μL of serial dilution was added to 100 μL of cell suspension to make final antibody concentrations of 10, 3, 1, 0.3, 0.1, 0.03, and 0 μg/mL, respectively. After 30 minutes of incubation on ice, each tube of cell suspension was added 1 mL of FACS working solution, the mix cells were whirled and centrifuged for 5 minutes at 1200 rpm/min, the supernatant was discarded, and repeated the wash. FITC-labeled goat anti-human IgG Fc antibody (Jackson ImmunoResearch Lab) was diluted with FACS working solution. Each tube of cell suspension was added 10 μL of antibody to final concentration of 1 μg/mL, protected from light, and incubated on ice for 30 min. After the incubation was completed, each tube of cell suspension was added 1 mL of FACS working solution, vortex to mix the cells, centrifuged for 5 minutes at 1200 rpm/min, discarded the supernatant, and repeated the wash. Cells were detected with flow cytometry C6 (BD Biosciences).

### Results & Analysis

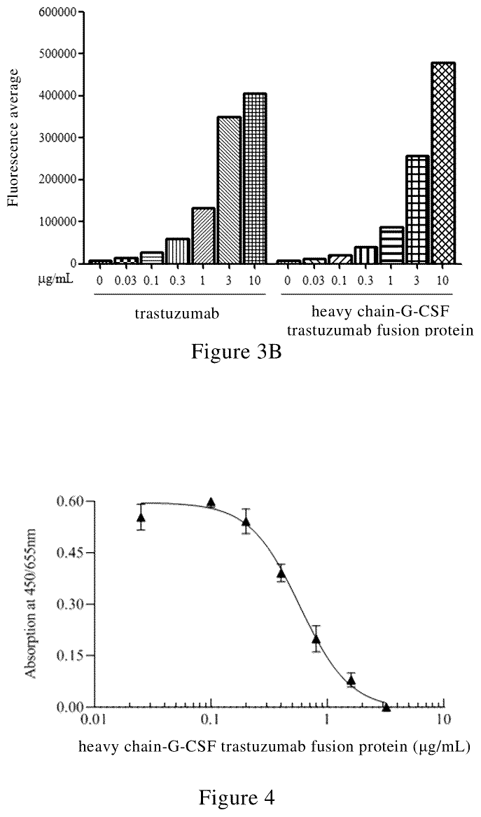

The results of the ELISA assay (FIG. 3A) showed that the heavy chain-G-CSF trastuzumab fusion protein specifically binds to Her-2 ECD with an EC₅₀ of 72 ng/mL.

Flow cytometry experiment showed that the heavy chain-G-CSF trastuzumab fusion protein can bind to Her-2 on the cell membrane, and the binding capacity was comparable to trastuzumab, as shown in FIG. 3B.

The fusion protein of the present invention perfectly retains the binding property of trastuzumab and Her-2.

### Example 5. Effect of Trastuzumab-G-CSF Fusion Protein on the Growth of her-2-Positive Human Breast Cancer BT-474 Cells

Trastuzumab inhibits the growth of Her-2-positive human tumor cells in vitro, such as human breast cancer cells BT-474. The effect of trastuzumab-G-CSF fusion protein on the growth of BT-474 cells in vitro was studied. BT-474 cells were cultured in RPMI1660 medium containing 10% FBS. BT-474 cells were cultured in 96-well plates for 1 day, then, added trastuzumab-G-CSF fusion protein of serial dilutions and continue cultured for 5 days. The cell viability assay reagent CCK-8 was added and the plate was read at a dual wavelength of 450 nm/655 nm by a microplate reader.

### Results & Analysis

The results showed that heavy chain-G-CSF trastuzumab inhibits the growth of Her-2-positive human breast cancer cells BT-474 in vitro, with an inhibitory IC₅₀ of 0.58 μg/mL. See FIG. 4.

### Example 6. Study of the G-CSF Cells Biological Activity of Trastuzumab-G-CSF Fusion Protein

The growth of mouse myeloid leukemia lymphocytes (NFS-60) is dependent on G-CSF, and the effect of trastuzumab-G-CSF fusion protein on the growth of NFS-60 cells in vitro was studied. NFS-60 cells were obtained from the Cell Bank of the Typical Culture Collection Committee of the Chinese Academy of Sciences and cultured in RPMI1640/10% FBS (Gibco) medium. NFS-60 cells were cultured in 96-well plates for 1 day, and trastuzumab-G-CSF fusion protein or recombinant human G-CSF (rhG-CSF) of serial dilutions were added and cultured for 3 days. Then the cell viability assay reagent CCK-8 was added and the plate was read at a dual wavelength of 450 nm/655 nm by a microplate reader.

### Results & Analysis

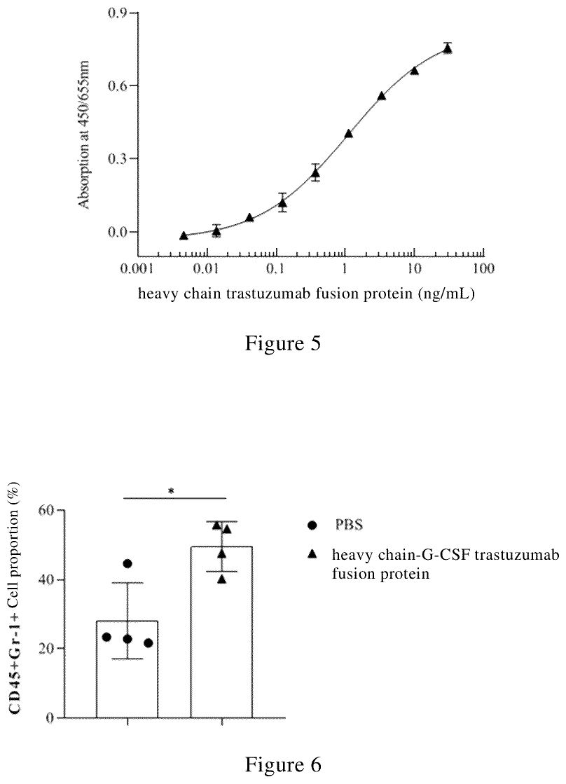

The results showed that heavy chain-G-CSF trastuzumab can stimulate the growth of NFS-60 cells in vitro, and the activity of stimulating cell growth EC₅₀ was 6.0 pmole/L, which was comparable to recombinant human G-CSF (4.6 pmole/L). See FIG. 5.

### Example 7. Study of Trastuzumab-G-CSF Fusion Protein Stimulate Neutrophil Proliferation in Mice

Eight C57BL/6 mice (10 to 12-week-old) were divided into two groups, and were given PBS and 2.5 mg/kg doses of heavy chain-G-CSF trastuzumab fusion protein, respectively, and blood was collected 72 hours later and placed in a test tube containing anticoagulant. Then, the FITC fluorescently labeled anti-mouse CD45 antibody and PE fluorescently labeled anti-mouse Gr-1 antibody was added, the fluorescent antibody-bound blood cells were analyzed by flow cytometry.

### Results & Analysis

The content of neutrophils (Gr-1 positive) in the mice blood of the PBS group was about 28%, while the content of neutrophils in the mice blood of the heavy chain-G-CSF trastuzumab fusion protein group was about 50%, indicating that the heavy chain-G-CSF trastuzumab fusion protein can promote the proliferation of neutrophils in the mice blood (see FIG. 6).

### Example 8. Construction of Mouse Tumor Cells Stably Expressing Human her-2

Mouse melanoma cells B16 were obtained from the Cell Bank of the Typical Culture Collection Committee of the Chinese Academy of Sciences and cultured in RPMI1640/10% FBS (Gibco) medium.

The human Her-2 expression gene was cloned in the expression vector pcDNA3.1 (Invitrogen), the recombinant plasmid was transfected with Lipofectmaine 3000 (Invitrogen) into mouse melanoma cells B16, and the transfected cells were cultured in RPMI/10% FBS medium containing G418 (Sigma) to obtain a stable cell pool. Monoclonal stable cell line B16/Her-2 expressing human Her-2 was selected from the stable cell pool by flow cytometry (Influx, BD Biosciences).

### Example 9. Trastuzumab-G-CSF Fusion Protein Inhibits the Growth of Human her-2-Expressing Mouse Melanoma B16 in Mice

C57BL/6 mice were bred in an SPF environment from Shanghai Slack Co., Ltd.

Thirty-two C57BL/6 mice (6-7-week-old) were divided into 4 groups of 8 mice with half male and half female. 1×10⁶ B16/Her-2 cells were injected into the axillary area of mice by subcutaneous vaccination. Eight days after inoculation of cells (D8), mice were given PBS (control group) or trastuzumab-G-CSF fusion protein 10 mg/kg or trastuzumab 10 mg/kg or trastuzumab 8 mg/kg in combination with trastuzumab and rhG-CSF 2 mg/kg (the mass ratio of trastuzumab to G-CSF in the trastuzumab-G-CSF fusion protein molecule was 4:1) in the tail vein, twice a week for a total of 4 doses. The tumor volume and mice weight were measured at each dose. At the end of the experiment on the 23rd day after inoculation of cells, the mice were sacrificed by neck dislocation, the blood were taken from eyeballs, the blood of the mice was collected, the mice were dissected, the weight of the tumor and the weight of the spleen were recorded, and the photos of the tumors in each group were recorded.

### Results & Analysis

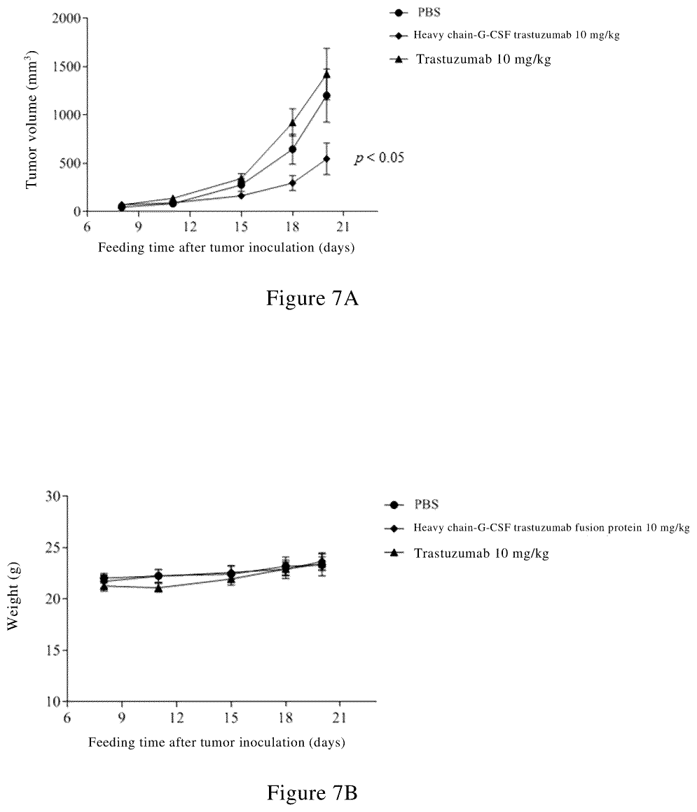

FIG. 7 shows that at D20, compared to the average tumor volume of the control group mice, the relative proliferation rate of mice in the heavy chain-G-CSF trastuzumab fusion protein group was 30%, significantly inhibiting the growth of B16/Her-2 tumors in mice. Trastuzumab had a little effect on tumor growth at a dose of 10 mg/kg. Experiments demonstrated that the inhibition effect of heavy chain-G-CSF trastuzumab fusion protein on B16/Her-2 tumor growth in vivo was much better than trastuzumab.

At the same time, the toxicity of trastuzumab-G-CSF fusion protein in mice was assessed with changes in mouse body weight. The results showed that there was no significant difference in the change of the average body weight of mice in the three groups, indicating that the heavy chain-G-CSF trastuzumab fusion protein had no significant toxicity to mice.

### Example 10. Effect of Trastuzumab-G-CSF Fusion Protein on Tumor Growth of her-2-Positive Human Gastric Cancer Cells NCI-N87 in Mice

Nude mice were bred in an SPF environment from Shanghai Slack Co., Ltd.

Thirty-two nude mice (6-7-week-old) were divided into 3 groups of 8 in each group, half male and half female. NCI-N87 cells were injected into the axillary area of mice with 1×10⁶ cells/mouse by subcutaneous inoculation. Eight days after inoculation of cells (D8), mice were given PBS (control group) or trastuzumab-G-CSF fusion protein 10 mg/kg or trastuzumab 10 mg/kg or trastuzumab 8 mg/kg in combination with trastuzumab and rhG-CSF 2 mg/kg (the mass ratio of trastuzumab to G-CSF in the trastuzumab-G-CSF fusion protein molecule was 4:1) in the tail vein, twice a week for a total of 4 doses. The tumor volume and mice weight were measured at each dose. The experiment ended on 23 days after the cells were inoculated, the mice were sacrificed by neck dislocation, the blood were taken from the eyeballs, the blood of the mice was collected, the mice were dissected, the weight of the tumor and the weight of the spleen were recorded, and the photos of the tumors in each group were recorded.

All literatures mentioned in the present application are incorporated herein by reference, as though each one is individually incorporated by reference. In addition, it should also be understood that, after reading the above teachings of the present invention, those skilled in the art can make various changes or modifications, equivalents of which falls in the scope of claims as defined in the appended claims.

1. A fusion protein single strand, wherein the fusion protein single strand comprises the following elements fused together:
(a) first protein element;
(b) second protein element;
(c) optional linker element between the first and the second protein elements;
wherein, the first protein element is an antigen recognition module;
the second protein element is granulocyte colony-stimulating factor.

2. A fusion protein consisting of the fusion protein single strand of claim 1, wherein the fusion protein comprises two single strands, each of which from the N-terminus to the C-terminus has a structure as shown in the following equation I: 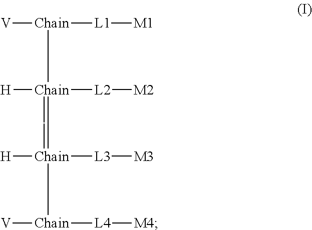
wherein M1, M2, M3 and M4 were independently none or granulocyte colony-stimulating factor G-CSF, and at least one of them was not none;
wherein L1, L2, L3, and L4 are independently none or bond or peptide linkers;
wherein 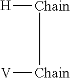
is a protein element of an anti-Her-2 antibody or its active fragment,
wherein
H-Chain is an anti-Her-2 antibody heavy chain or its active fragment;
V-Chain is an anti-Her-2 antibody light chain or its active fragment;
wherein [symbol] denotes the disulfide bond between the heavy and light chains; and
wherein “−” denotes the peptide bond.

3. The fusion protein of claim 2, wherein the fusion protein is homologous or heterodimer.

4. The fusion protein of claim 2, wherein the length of the peptide linker is 0-20 amino acids, preferably 1-15 amino acids.

5. The fusion protein of claim 2, wherein the H-Chain comprises or has amino acids on positions 1-449 in SEQ ID NO: 11, the V-Chain comprises or has amino acids on positions 1-214 in SEQ ID NO: 14, and the G-CSF contains or has amino acids on positions 450-624 in SEQ ID NO: 11, or amino acids on positions 222-396 in SEQ ID NO: 14.

6. The fusion protein of claim 2, wherein the sequence of the fusion protein is selected from the following group:
(1) the sequence of H-chain-M as shown in SEQ ID NO:11 and the sequence of V-chain as shown in SEQ ID NO: 12;
(2) the sequence of H-chain as shown in SEQ ID NO:13 and the sequence of V-chain-M as shown in SEQ ID NO:14;
(3) derived sequences having ≥ 80% homology to the above sequences (preferably, ≥90% homology; more preferably ≥95% homology; most preferably, ≥97% homology, such as more than 98%, more than 99%).

7. An isolated polynucleotide, wherein the polynucleotide encoding a fusion protein of claim 2.

8. A vector, wherein the vector contains the polynucleotide of claim 7.

9. A host cell, wherein the host cell contains the vector of claim 5.

10. A method for producing the fusion protein of claim 2, which comprises steps:
(1) under conditions suitable for expression, culturing the host cells of claim 9, thereby expressing the fusion protein of claim 2; and
(2) optionally isolating the fusion protein.

11. A pharmaceutical composition, wherein the composition comprises:
the fusion protein of claim 2, and
pharmaceutically acceptable carriers.

12. An immune cell, wherein the immune cell carries a fusion protein of claim 2.

13. A pharmaceutical composition, wherein the composition comprises:
the immune cells of claim 12, and
pharmaceutically acceptable carriers.

14. A method for preventing and/or treating a tumor, comprising steps: administering the fusion protein of claim 2 to a subject in need.

15. The method of claim 14, wherein the tumor including breast cancer tumors, gastric cancer tumors, bladder cancer tumors, pancreatic cancer tumors, colorectal cancer tumors, lung cancer tumors, liver cancer tumors, and melanoma tumors.
